(19) United States
(12) Patent Application Publication
(10) Pub. No.: US 2024/0120036 A1
(43) Pub. Date: Apr. 11, 2024

(54) PROCESSING APPARATUS, SYSTEM, METHOD, AND PROGRAM FOR CALCULATING A STRUCTURAL FACTOR

(71) Applicant: Rigaku Corporation, Tokyo (JP)

(72) Inventors: Masatsugu YOSHIMOTO, Tokyo (JP); Kazuhiko OMOTE, Tokyo (JP); Kazuki ITO, Tokyo (JP)

(73) Assignee: Rigaku Corporation, Tokyo (JP)

(21) Appl. No.: 18/373,967

(22) Filed: Sep. 28, 2023

(30) Foreign Application Priority Data
Sep. 30, 2022 (JP) 2022-157711

(51) Int. Cl. G16C 60/00; G01N 23/20016

(52) U.S. Cl. CPC G16C 60/00; G01N 23/20016

(57) ABSTRACT

A processing apparatus for processing a structure factor including total scattering data and data of a structural model are provided comprises a structure factor acquiring section for acquiring a first structure factor based on measured total scattering data; a data converting section for separating the first structure factor into a short-range correlation and a long-range correlation; and a scattering intensity calculating section for acquiring a structural model indicating an atomic arrangement in a finite region, calculating a short-range scattering intensity of the structural model and calculating a second structure factor from the short-range scattering intensity and the long-range correlation.

### CROSS REFERENCE TO RELATED APPLICATIONS

This application claims priority from Japanese Patent Application No. 2022-157711, filed on Sep. 30, 2022, the entire contents of which are incorporated by reference in this application.

### BACKGROUND

### Field

The present disclosure relates to a processing apparatus, a system, a method, and a program for processing a structural factor.

### Description of the Related Art

In order to deeply understand materials functions, three-dimensional structure information is indispensable. Many of conventional materials are crystalline materials, and thus objectives have been able to be achieved by determining crystal structures. However, in recent years, in materials such as batteries and materials in electronic fields, there are also many crystalline materials in which regularity is positively lowered in order to maximize the function and physical properties of the object.

Conventionally, localized structure estimation of crystalline materials has required complicated setting of parameters by the user to calculate diffraction peaks. Therefore, there is a need for a method for estimating a local structure of a crystalline material that does not require complicated setting of parameters.

Non-Patent Document 1 discloses methods for calculating diffracted peaks using RMCPOW method. Non-Patent Document 2 discloses a process for calculating diffracted peaks using RMCProfile method. Patent Document 1 discloses a method for deriving a crystalline structural model and structural parameters for reproducing the PDF by measured values.

### Non-Patent Document

Non-patent Document 1: A. Mellergård, R. L. McGreevy, Acta Crystallogr Acta Crystallogr. 55 (1999) 783-789.
Non-patent Document 2: M. G. Tucker, M. T. Dove, D. A. Keen, J. Appl. Crystallogr. 34 (2001) 630-638.

### Patent Document

Patent Document 1: JP-A-2020-94945

However, Non-Patent Document 1 does not describe how to set the resolution function of the scattering vector Q, and it is necessary for the user to appropriately set a parameter in which the processing proceeds successfully. In Non-Patent Document 2, the parameter of the profile-function has to be calculated separately using software called GSAS. This operation has to be performed via a converter, which takes calculation cost similar to performing a normal Rietveld analysis.

Further, in the method described in Patent Document 1, parameters having wave number dependency such as a resolution function and an atomic scattering factor would be strictly handled, and therefore, it is necessary for the user to set various parameters, which takes calculation cost. That is, in all the methods of Non-Patent Document 1, Non-Patent Document 2, and Patent Document 1, many complicated parameters need to be set by the user, and it is not easy to generate a structural model that can explain actual measurement data. In addition, calculation costs are incurred.

### SUMMARY

As a result of intensive research, the present inventors have found that by calculating a structure factor including measured total scattering data and data of a structural model, parameters set by a user when estimating a local structure of a sample can be simplified, that calculation costs can be reduced, that characteristics of both the total scattering data and the structural model can be analyzed together, and that further, a highly accurate structural model capable of explaining the measured data can be generated, and the present disclosure has been completed.

The present disclosure has been made in view of such circumstances and provides a processing apparatus, a system, a method, and a program for calculating a structure factor including total scattering data and data of a structural model.

(1) The processing apparatus of the present disclosure is a processing apparatus for processing a structure factor comprises a structure factor acquiring section for acquiring a first structure factor based on measured total scattering data; a data converting section for separating the first structure factor into a short-range correlation and a long-range correlation; and a scattering intensity calculating section for acquiring a structural model indicating an atomic arrangement in a finite region, calculating a short-range scattering intensity of the structural model and calculating a second structure factor from the short-range scattering intensity and the long-range correlation.

(2) Further, in the processing apparatus of the present disclosure, a value of a boundary between the short-range correlation and the long-range correlation is determined based on a size and a shape of a region of the structural model.

(3) Further, the processing apparatus of the present disclosure further comprises a structure evaluating section for calculating a degree of coincidence or a degree of deviation between the first structure factor and the second structure factor.

(4) Further, the processing apparatus of the present disclosure further comprises a structure estimating section for creating the structural model, wherein the structure evaluating section outputs the structural model in which the degree of coincidence or the degree of deviation satisfies a predetermined condition.

(5) Further, in the processing apparatus of the present disclosure, the structure evaluating section calculates the degree of coincidence or deviation between the first structure factor and the second structure factor within a range equal to or greater than a lower limit value determined based on the value of the boundary between the short-range correlation and the long-range correlation.

(6) Further, in the processing apparatus of the present disclosure, the structure estimating section generates the structural model by an RMC method.

(7) Further, the processing apparatus of the present disclosure further comprises a structure factor calculating section for acquiring total scattering data of the sample and calculating the first structure factor based on a type of a radiation source, a wavelength, a background, a shape of the sample, an arrangement, kinds of a constituent element, a composition, and an absorption coefficient of the total scattering data, wherein the structure factor acquiring section acquires the first structure factor calculated by the structure factor calculating section.

(8) Further, the system of the present disclosure, comprises an X-ray diffractometer comprises an X-ray generating section for generating X-rays, a detector for detecting X-rays, and a goniometer for controlling the rotation of the sample and the processing apparatus described in any of (1) to (7).

(9) Further, the method of the present disclosure is a method for processing a structure factor, comprises the steps of acquiring a first structure factor based on measured total scattering data, separating the first structure factor into a short-range correlation and a long-range correlation, acquiring a structural model representing an atomic arrangement in a finite region, calculating a short-range scattering intensity of the structural model, and calculating a second structure factor from the short-range scattering intensity and the long-range correlation.

(10) Further, the program of the present disclosure is a program for processing a structure factor and causes a computer to perform the following processing of acquiring a first structure factor based on measured total scattering data, separating the first structure factor into a short-range correlation and a long-range correlation, acquiring a structural model representing an atomic arrangement in a finite region, calculating a short-range scattering intensity of the structural model, and calculating a second structure factor from the short-range scattering intensity and the long-range correlation.

### BRIEF DESCRIPTION OF THE DRAWINGS

FIG. 1 is a graph showing an example of a first structure factor F_{obs} (Q).

FIG. 2 is a graph showing an example of a first structure factor F_{obs} (Q), a short-range correlation F^{S}_{obs} (Q) and a long-range correlation F^{L}_{obs} (Q).

FIG. 3 is a graph showing an example of a short-range scattering intensity F^{S}_{cal} (Q) of a structural model.

FIG. 4 is a graph showing an example of a first structure factor S_{obs} (Q), a second structure factor S_{cal} (Q), and a residual thereof.

FIG. 5 is a schematic diagram showing an example of an output structural model.

FIG. 6 is a schematic diagram showing an example of a configuration of the X-ray diffraction measuring system.

FIG. 7 is a block diagram showing an example of a configuration of the control apparatus.

FIG. 8 is a block diagram showing a modified example of a configuration of the control apparatus and the processing apparatus.

FIG. 9 is a block diagram showing a modified example of a configuration of the control apparatus and the processing apparatus.

FIG. 10 is a block diagram showing a modified example of a configuration of the processing apparatus.

FIG. 11 is a block diagram showing a modified example of a configuration of the processing apparatus.

FIG. 12 is a block diagram showing a modified example of a configuration of the processing apparatus.

FIG. 13 is a block diagram showing a modified example of a configuration of the processing apparatus.

FIG. 14 is a flowchart showing an example of the operation of the processing apparatus.

FIG. 15 is a flowchart showing a modified example of the operation of the processing apparatus.

FIG. 16 is a flowchart showing a modified example of the operation of the processing apparatus.

FIG. 17 is a graph showing PDF and residuals of actual measurement value, examples, and comparative examples.

FIG. 18 is a histogram of displacement amounts calculated from a structural model generated with the method of the present disclosure.

### DETAILED DESCRIPTION

Next, embodiments of the present disclosure are described with reference to the drawings. To facilitate understanding of the description, the same reference numerals are assigned to the same components in the respective drawings, and duplicate descriptions are omitted.

### [Principle]

As a local structure estimation method of the sample, there is RMC (Reverse Monte Carlo) method. RMC method is a method for estimating a structural model that reproduces an actual measurement value by moving atoms arranged by a given structural model using a random number. Local structure estimation by RMC method is based on the assumption that the crystal phase of the material for estimating the local structure is known and the value of the parameter for determining the width of the diffraction peak is known, and the parameters and the resolution function of Q for calculating the diffraction peak has to be set by the user every time.

In addition, in order to calculate the diffracted peak from the structural model, it is necessary to calculate the total scattering data from the structural model, and that takes the capacity of memory and CPU and the like in the computer and the calculation cost such as the computational time.

Since the method of the present disclosure does not calculate the diffraction peak directly from the structural model, it is not necessary for the user to set a parameter for calculating the diffraction peak and a resolution function of the scattering vector Q. Further, since only the short-range scattered data is calculated from the structural model, it is not necessary to calculate the total scattering data including the long-range correlation, and the calculation cost can be reduced.

In the method of the present disclosure, first, measured total scattering data is acquired and generated a first structure factor. As the total scattering data, for example, total scattering data by means of X-rays, total scattering data by means of synchrotron radiation, and total scattering data by means of particle beams such as neutron beams and electron beams can be used. A structure factor is defined as a Fourier transform of the spatial correlation of the electron density distribution (or nuclear density distribution) in a material and is a value used in determining the elastic scattering or coherent scattering intensity. The first structure factor is a structure factor generated from the measured total scattering data. Next, the first structure factor is separated into a short-range correlation and a long-range correlation. The short-range correlation and the long-range correlation are obtained by separating a correlation function on a real space obtained by Fourier transforming a structure factor by a value of a predetermined boundary. Therefore, the value of the boundary between the short-range correlation and the long-range correlation is the value of the dimension of the distance.

Next, a structural model showing the atomic arrangement in the finite region is generated and acquired, and the short-range scattering intensity of the structural model is calculated. The structural model is data indicating an atomic arrangement in a finite region, and indicates, for example, an arrangement of finite number of atoms in a cube, cuboid, or parallel hexahedron. The short-range scattering intensity is a scattering intensity calculated from the atomic arrangement in the finite region. In order to reproduce the total scattering data measured from the structural model, a large structural model is required because calculation including long-range correlation is required. On the other hand, since the short-range scattering intensity does not include long-range correlation, the calculation is possible even with a small structural model. Since the size of the structural model is correlated with the calculation cost, the calculation cost for calculating the short-range scattering intensity is smaller than the calculation cost for performing calculations to reproduce the measured total scattering data. That is, the present disclosure can reduce the calculation cost compared to the conventional technique.

Next, the total scattering data and a second structure factor including the data of the structural model are calculated from the short-range scattering intensity of the structural model and the long-range correlation of the first structure factor. The second structure factor is a structure factor that includes both the measured total scattering data and the data of the structural model. Next, the degree of coincidence or deviation between the first structure factor and the second structure factor is calculated. The degree of coincidence or deviation between the first structure factor and the second structure factor is an index indicating the degree of similarity between the first structure factor and the second structure factor. Then, when the degree of coincidence or deviation does not satisfy the predetermined condition, the structural model is generated again, and the second structure factor is calculated. When the degree of coincidence or deviation satisfies the predetermined condition, the process ends.

Since the second structure factor calculated as described above includes both the measured total scattering data and the data of the structural model, it is possible to confirm how much the given structural model reproduces the measured total scattering data by analyzing the data. In addition, a structural model in which the degree of coincidence or deviation satisfies a predetermined condition can be said to be a highly accurate structural model that can explain the actual measurement data. The detailed processing method according to the present disclosure is detailed in the embodiment.

### Embodiment

The processing method according to the present disclosure is explained in detail, as described below. Hereinafter, a method of processing a first structure factor based on total scattering data measured by an X-ray diffractometer and calculating a second structure factor including total scattering data and data of a structural model, a method of calculating a degree of coincidence or deviation between the first structure factor and the second structure factor, and a method of outputting a structural model in which the degree of coincidence or deviation is a predetermined threshold or less are described. However, the total scattering data to which the present disclosure can be applied is not limited to the total scattering data measured by an X-ray diffractometer and be applied to the total scattering data measured by a probe similar thereto. For example, it can be applied to the total scattering data by means of synchrotron radiation and to the total scattering data by means of particle beams such as neutron beams and electron beams. Further, the present disclosure does not necessarily require acquisition of the total scattering data, and thus the first structure factor calculated from the total scattering data may be acquired as first data.

First, the total scattering data measured by the X-ray diffractometer is acquired. In one aspect, when the total scattering data is used as the first data, information required for calculating the structure factor can also be acquired, based on the total scattering data such as a type of a radiation source, a wavelength, a background, a shape of a sample, arrangement, kinds of constituent elements, a composition, an absorption coefficient or the like, in the total scattering data. These pieces of information may be stored in advance or acquired from an X-ray diffractometer. Further, they may be input by a user.

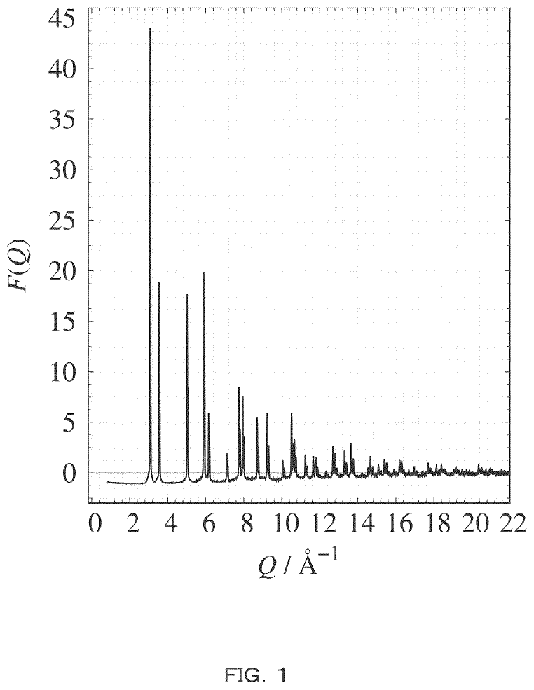

Next, the first structure factor F_{obs} (Q) is calculated based on the total scattering data. The calculation of the first structure factor F_{obs} (Q) is based on the type of the radiation source, the wavelength, the background, the shape of the sample, the arrangement, the kinds of constituent elements, the composition, the absorption coefficient or the like, in the total scattering data. FIG. 1 is a graphical showing an example of the first structure factor F_{obs} (Q).

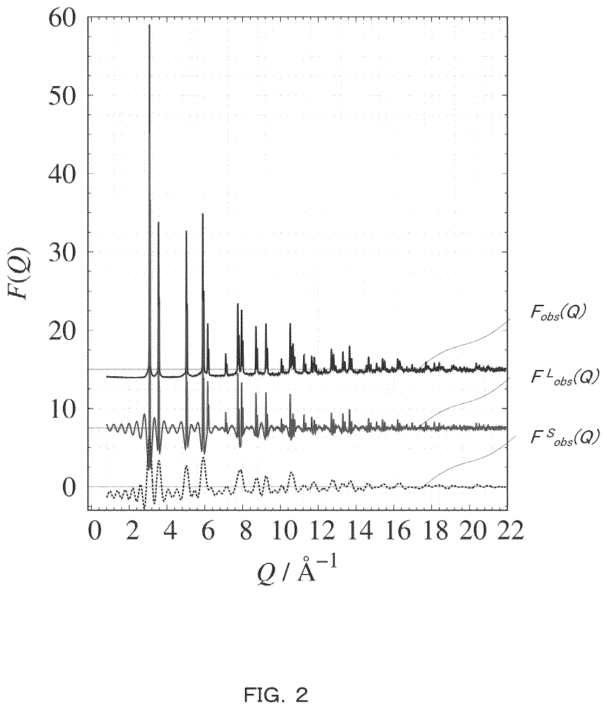

Next, the first structure factor F_{obs} (Q) is separated into a short-range correlation F^{S}_{obs} (Q) and a long-range correlation F^{L}_{obs} (Q). The first structure factor F_{obs} (Q) is expressed by the following Formula (1) using the short-range correlation F^{S}_{obs} (Q) and the long-range correlation F^{L}_{obs} (Q). FIG. 2 is a graph showing an example of the first structure factor F_{obs} (Q), the short-range correlation F^{S}_{obs} (Q) and the long-range correlation F^{L}_{obs} (Q). The short-range correlation F^{S}_{obs} (Q) and the long-range correlation F^{L}_{obs} (Q) in FIG. 2 are plots separating the first structure factors F_{obs} (Q) in FIG. 1.

F_{obs}(Q)=F_{obs}^{S}+F_{obs}^{L}(Q) (1)

In one aspect, value of a boundary between the short-range correlation F^{S}_{obs} (Q) and the long-range correlation F^{L}_{obs} (Q) is determined based on the size and shape of the regions of the structural model. For example, when the radius of the largest sphere included in the structural model is expressed as a rₘₐₓ, rₘₐₓ may be the value of the border between the short-range correlation F^{S}_{obs} (Q) and the long-range correlation F^{L}_{obs} (Q). Hereinafter, r max is used as the value of the border between the short-range correlation F^{S}_{obs} (Q) and the long-range correlation F^{L}_{obs} (Q), but other values may be used.

Any method may be used to separate the first structure factor F_{obs} (Q) into a short-range correlation F^{S}_{obs} (Q) and a long-range correlation F^{L}_{obs} (Q). For example, that is conveniently performed by using PDF (Pair Distribution Function) G_{obs} (r) obtained by Fourier-transforming the first structure factor F_{obs} (Q). Calculation of G_{obs} (r) is performed by obtaining the smallest value Qₘᵢₙ and the largest value Qₘₐₓ of the first structure factor F_{obs} (Q) by the following Formula (2). Qₘᵢₙ and Qₘₐₓ are associated with calculating the first structure factor F_{obs} (Q). Qₘᵢₙ and Qₘₐₓ may be entered by the user.

$${G}_{obs} ( r ) = \frac{2}{π} {\int }_{{Q}_{min}}^{{Q}_{max}} {Q}^{2} {F}_{obs} ( Q ) \frac{sin Qr}{Qr} dQ \qquad ( 2 )$$

As shown in the following Formula (3), the first structure factor F_{obs} (Q) is obtained by inverse transforming G_{obs} (r). Therefore, when the value of the border between the short-range correlation F^{S}_{obs} (Q) and the long-range correlation F^{L}_{obs} (Q) is assumed rₘₐₓ, the first structure factor F_{obs} (Q) can be separated as shown in the following Formula (4).

$${F}_{obs} ( Q ) = {\int }_{0}^{∞} {G}_{obs} ( r ) \frac{sin Qr}{Qr} dr \qquad ( 3 )$$

$${F}_{obs} ( Q ) = {\int }_{0}^{{r}_{max}} {G}_{obs} ( r ) \frac{sin Qr}{Qr} dr + {\int }_{{r}_{max}}^{∞} {G}_{obs} ( r ) \frac{sin Qr}{Qr} dr \qquad ( 4 )$$

Therefore, when the first structure factor F_{obs} (Q) is separated using G_{obs} (r), for example, the short-range correlation F^{S}_{obs} (Q) can be defined by the following Formula (5). Further, using the short-range correlation F^{S}_{obs} (Q) obtained by Formula (5), the long-range correlation F^{L}_{obs} (Q) can be obtained by the following Formula (6).

$${F}_{obs}^{S} ( Q ) = {\int }_{o}^{{r}_{max}} {G}_{obs} ( r ) \frac{sin Qr}{Qr} dr \qquad ( 5 )$$

$${F}_{obs}^{L} ( Q ) = {F}_{obs} ( Q ) - {F}_{obs}^{S} ( Q ) \qquad ( 6 )$$

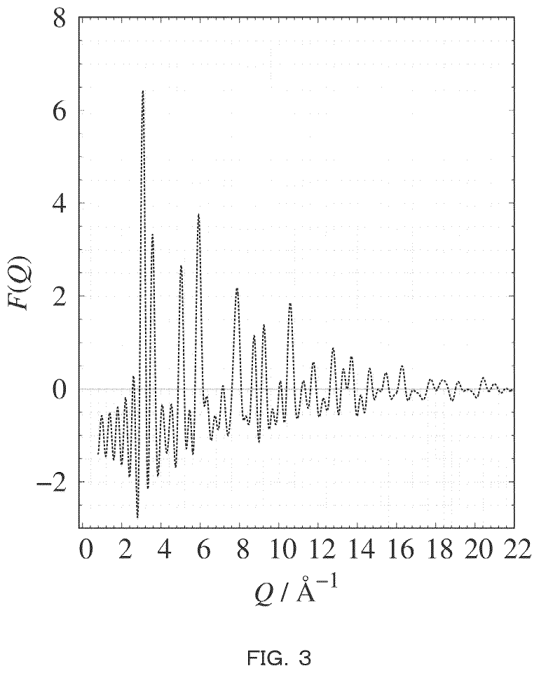

Next, a structural model showing the atomic arrangement in the finite area is obtained, and a short-range scattering intensity F^{S}_{cal} (Q) of the structural model is calculated. The structural model can be given as data indicating the arrangement of a finite number of atoms in, for example, a cube, a cuboid, or a parallel hexahedron, depending on the sample. The short-range scattering intensity F^{S}_{cal} (Q) of the structural model can be calculated, for example, by the following Formula (7). In Formula (7), N is the number of atoms in the structural model. rᵢⱼ is defined by Formula (8) when the i-th atomic arrangement is expressed as nᵢ (xᵢ, yᵢ, zᵢ) and the j-th atomic arrangement is expressed as nⱼ (xⱼ, yⱼ, zⱼ) with respect to the atomic arrangement n (x,y,z) of the structural model. fᵢ and fⱼ are the i-th and j-th atomic scattering factors, respectively. Q is a scattering vector. FIG. 3 is a graph showing an example of the short-range scattering intensity F^{S}_{cal} (Q) of the structural model.

$${F}_{cal}^{S} ( Q ) = \frac{1}{N} {\sum }_{i}^{N} {\sum }_{j \neq i}^{N} {f}_{i} {f}_{j} \frac{sin {Qr}_{ij}}{{Qr}_{ij}} \qquad ( 7 )$$

$${r}_{ij} = \sqrt{{( {x}_{j} - {x}_{i} )}^{2} + {( {y}_{j} - {y}_{i} )}^{2} + ( {z}_{j} - {z}_{i} )} \qquad ( 8 )$$

Then, the second structure factor F_{cal} (Q) including the total scattering data and the data of the structural model is calculated from the short-range scattering intensity F^{S}_{cal} (Q) of the structural model and the long-range correlation F^{L}_{obs} (Q) of the first structure factor. The second structure factor F_{cal} (Q) can be calculated, for example, by the following Formula (9). Thus, the features of both the total scattering data and the structural model can be analyzed together using the second structure factor F_{cal} (Q). Also, depending on the application of the second structure factor, the second structure factor may be calculated as S_{cal} (Q). The second structure factor S_{cal} (Q) can be calculated, for example, by the following Formula (10).

F_{cal}(Q)=F_{cal}^{S}(Q)+F_{obs}^{L}(Q) (9)

S_{cal}(Q)=F_{cal}^{S}(Q)+F_{obs}^{L}(Q)+1 (9)

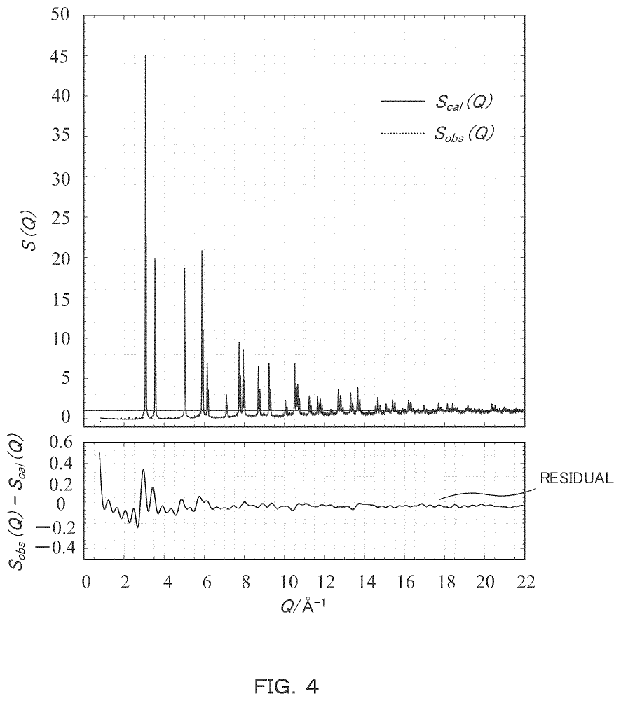

To confirm how accurate a given structural model reproduces the measured total scattering data, the degree of coincidence or deviation between the first structure factor F_{obs} (Q) and the second structure factor F_{cal} (Q) may be calculated. The degree of coincidence or deviation is calculated with the first structure factor F_{obs} (Q) and the second structure factor F_{cal} (Q) and may be any value as long as the value indicates the degree of similarity. The degree of similarity is larger when the value of coincidence is larger. The degree of similarity is larger when the value of deviation is smaller. The degree of deviation can be calculated, for example, by R_{P, S(Q)} of the following Formula (11). wᵢ in Formula (11) is a weighting factor, for example, being used as wᵢ=1/N. Also, S_{obs} (Q)=F_{obs} (Q)+1 and S_{cal} (Q)=F_{cal} (Q)+1 are held. As shown in Formula (11), the degree of coincidence or deviation between the first structure factor and the second structure factor may be calculated using S_{obs} (Q) or S_{cal} (Q). FIG. 4 is a graph showing an example of the first structure factor S_{obs} (Q) the second structure factor S_{cal} (Q) and the residual thereof. The mathematical expression for calculating the degree of coincidence or deviation is not limited to the Formula (11).

$${R}_{P , S ( Q )} = \sqrt{\frac{{\sum }_{i}^{N} {{w}_{i} ( {S}_{obs} ( {Q}_{i} ) - {S}_{cal} ( {Q}_{i} ) )}^{2}}{{\sum }_{i}^{N} {{w}_{i} ( {S}_{obs} ( {Q}_{i} ) )}^{2}}} \times 100 \qquad ( 11 )$$

When calculating the degree of coincidence or deviation between the first structure factor F_{obs} (Q) and the second structure factor F_{cal} (Q) in one aspect, the degree of coincidence or deviation within a range equal to or larger than the lower limit value determined based on the value of the border between the short-range correlation F^{S}_{obs} (Q) and the long-range correlation F^{L}_{obs} (Q) is calculated. G_{obs} (r) can be calculated from the measured first structure factor F_{obs} (Q) by the above Formula (2), which is a combination of the following Formulas (12) and (13). α(Q) is a step function and is an example of a function that cuts off the data on the short-range side and the long-range side of the first structure factor F_{obs} (Q)

$${G}_{obs} ( r ) = \frac{2}{π} {\int }_{0}^{∞} {Q}^{2} {F}_{obs} ( Q ) α ( Q ) \frac{sin Qr}{Qr} dQ \qquad ( 12 )$$

$$α ( Q ) = \{ \begin{matrix}1 , & {Q}_{min} \leq Q \leq {Q}_{max} \\ 0 , & 0 < Q < {Q}_{min} or {Q}_{max} < Q < ∞\end{matrix} \qquad ( 13 )$$

The effect of the step function appears in the first structure factor F_{obs} (Q), which is inverse transformed from PDF G_{obs}(r). The effect of the truncation error of Qₘᵢₙ is related to the value of the border of G_{obs} (r) (rₘₐₓ in Formula (4) above), and the resolution ΔQ′₀ of the Q of Qₘᵢₙ is expressed by the following Formula (14) using rₘₐₓ.

$$Δ {Q}_{0}^{′} = \frac{π}{{r}_{max}} \qquad ( 14 )$$

Therefore, by determining the following measured point Q′₁ of Qₘᵢₙ of the inversely transformed first structure factor F_{obs} (Q) as in the following Formula (15), the effect of the truncation error can be sufficiently reduced. That is, the degree of coincidence or deviation within a predetermined range equal to or larger than Q′₁ using the lower limit value determined based on the value of the border between the short-range correlation F^{S}_{obs} (Q) and the long-range correlation F^{L}_{obs} (Q) as a Q′₁ may be calculated. Further, Qₘᵢₙ may be a lower limit of the measured Q.

Q′₁=Qₘᵢₙ+ΔQ′₀ (15)

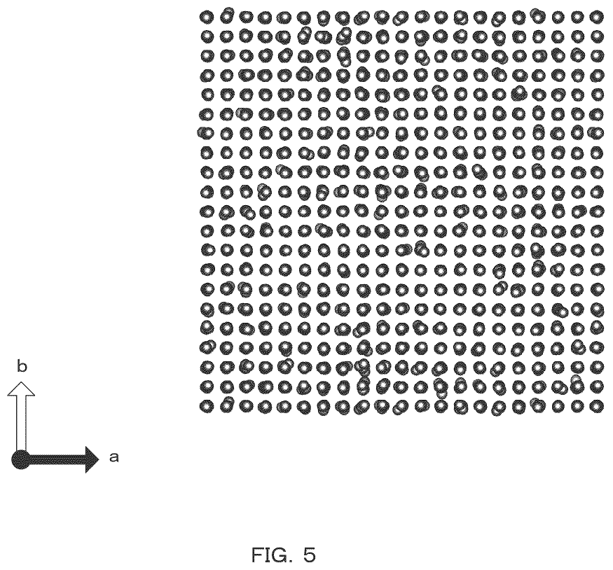

Hereinafter, the following describes how to generate a more appropriate structural model using the method of the present disclosure. It is assumed that the structural model can be generated repeatedly in some way. In one aspect, a structural model based on the degree of coincidence or deviation and output a structural model in which the degree of coincidence or deviation satisfies a predetermined condition is repeatedly generated. FIG. 5 is a schematic diagram showing an example of the output structural model. For example, in the case of using the degree of deviation in Formula (11), it is possible to adopt that the value of the degree of deviation is 5% or less as the predetermined condition.

When the structural model is repeatedly generated, any method may be used to generate the structural model. In one aspect, the structural model is generated by, for example, the RMC method. This is because the RMC method has a wide search space and can obtain a global minimum solution, which is useful as a solution for complicated optimization. Therefore, when RMC method is applied to the present disclosure it is highly likely that a structural model reproducing the measured scattered data will be obtained. In RMC method, the atomic arrangement of the structural model is moved randomly, and when the degree of coincidence or deviation after the operation is better than the degree of coincidence or deviation before the operation (the degree of similarity is larger), the atomic arrangement is further moved randomly. On the other hand, when the degree of coincidence or deviation after the operation is not better than the degree of coincidence or deviation before the operation (the degree of similarity is not larger), the operation is cancelled, and the random movement is performed again from the atomic arrangement before the operation. Such an operation is performed until the degree of coincidence or deviation satisfies a predetermined condition. Incidentally, the method for generating the structural model may be a MD method (Molecular Dynamics method) or a MC method (Monte Carlo method).

In this way, it is possible to generate a structural model that reproduces the measured total scattering data with sufficient accuracy.

### [Whole System]

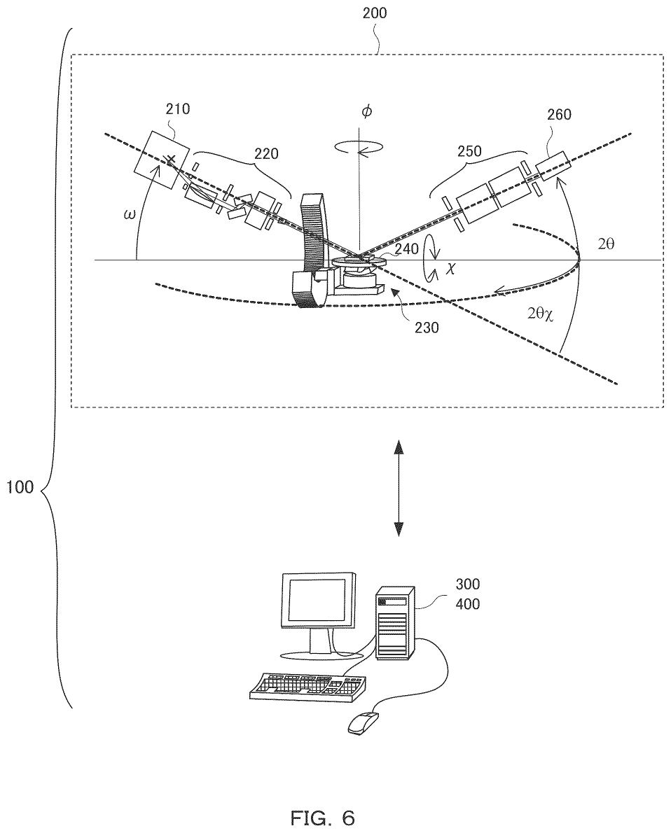

FIG. 6 is a schematic diagram showing an example of a configuration of an X-ray diffraction measurement system 100. The system 100 includes an X-ray diffractometer 200, a control apparatus 300, and a processing apparatus 400. The X-ray diffractometer 200 comprises an optical system for irradiating X-rays onto a sample and detecting diffracted X-rays generated from the sample, and the optical system comprises a goniometer. Incidentally, the configuration shown in FIG. 6 is one example, and thus a variety of other configurations may be adopted.

The control apparatus 300 is connected to the X-ray diffractometer 200 and controls the X-ray diffractometer 200 and processes and stores the acquired data. The processing apparatus 400 performs processing of a structure factor. The control apparatus 300 and the processing apparatus 400 are apparatuses including CPU and memories and may be PC terminals or servers on the cloud. Not only the whole apparatus but also part of the apparatus or some functions of the apparatus may be provided on the cloud. The input device 510 is, for example, a keyboard or a mouse, and performs input to the control apparatus 300 or the processing apparatus 400. The display device 520 is, for example, a display and displays structure factors, PDF, structural models, and the like.

Using such a system 100, the total scattering data can be measured, and the structure factors calculated from the total scattering data can be processed. In addition, a structural model can be generated, and a second structure factor including the total scattering data and data of the structural model can be calculated. As a result, the local structure of the sample can be estimated.

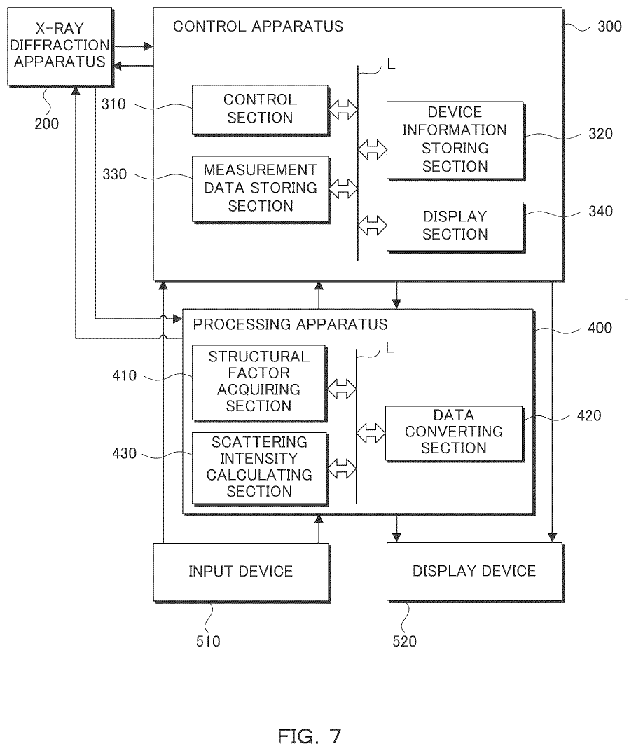

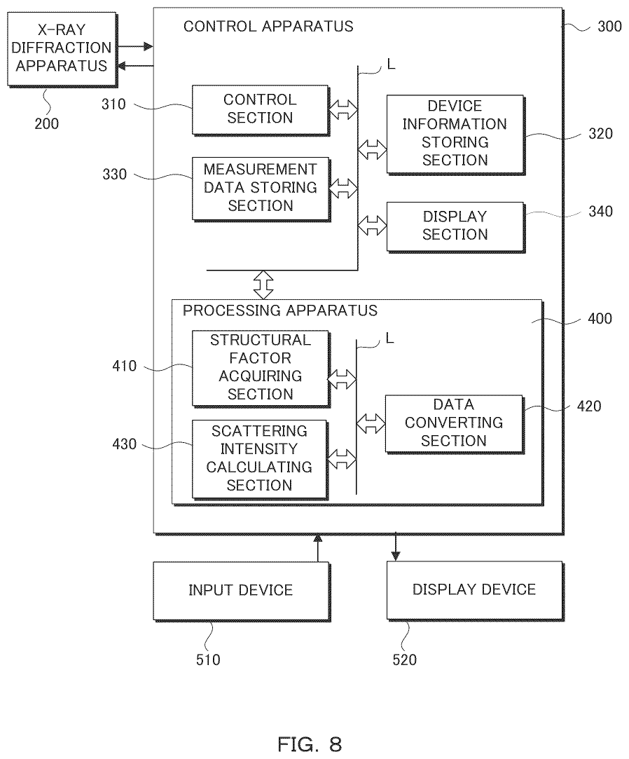

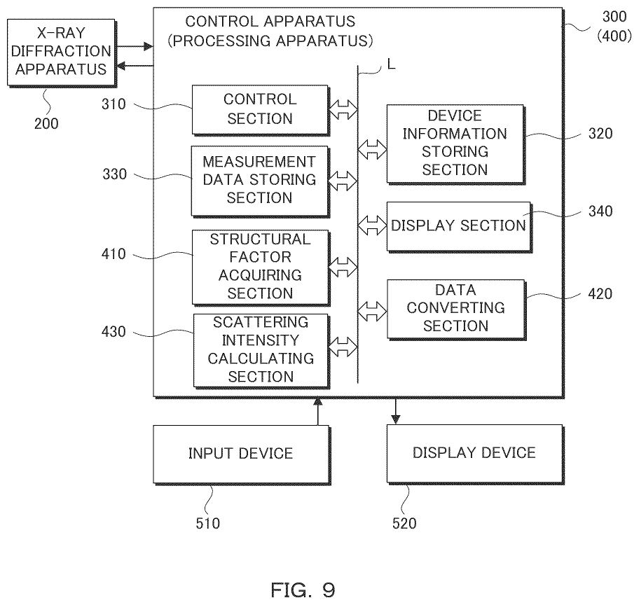

In FIG. 6, the control apparatus 300 and the processing apparatus 400 are described as the same PC. However, as explained above, in the method of the present disclosure, total scattering data or structure factors can be obtained and processed independent of the X-ray diffractometer 200 or the control apparatus 300. Therefore, as shown in FIG. 7, the processing apparatus 400 may be configured as an apparatus different from the control apparatus 300. FIG. 7 is a block diagram showing an example of a configuration of the control apparatus 300 and the processing apparatus 400. Further, as shown in FIG. 8, the processing apparatus 400 may be configured as a part of functions included in the control apparatus 300. As shown in FIG. 9, the processing apparatus 400 and the control apparatus 300 may be configured as an integrated apparatus. FIG. 8 and FIG. 9 are block diagrams showing modified examples of the configurations of the control apparatus 300 and the processing apparatus 400. Hereinafter, a case where the control apparatus 300 and the processing apparatus 400 are configured as different apparatuses is described.

### [X-Ray Diffractometer]

The X-ray diffractometer 200 comprises an X-ray generation section 210 that generates X-rays from an X-ray focus, that is, an X-ray source; an incident side optical unit 220; a goniometer 230; a sample table 240 where a sample is set; an emitting side optical unit 250; and a detector 260 that detects X-rays. The X-ray generation section 210, the incident side optical unit 220, the goniometer 230, the sample table 240, the emitting side optical unit 250, and the detector 260 each constituting the X-ray diffractometer 200 may be those generally available, and thus descriptions are omitted.

### [Control Apparatus]

The control apparatus 300 is constituted from a computer formed by connecting CPU (Central Processing Unit/Central Processor), ROM (Read Only Memory), RAM (Random Access Memory) and a memory to a bus. The control apparatus 300 is connected to the X-ray diffractometer 200 to receive information.

The control apparatus 300 comprises the control section 310, the apparatus information storage section 320, the measurement data storage section 330, and the display section 340. Each section can transmit and receive information via the control bus L. The input device 510 and the display device 520 are connected to CPU via an appropriate interface.

The control section 310 controls the operations of the X-ray diffractometer 200. The apparatus information storage section 320 stores apparatus information acquired from the X-ray diffractometer 200. The apparatus information includes information about the X-ray diffractometer 200 such as name of the apparatus, the kind of a radiation source, a wavelength, a background, and so forth. In addition, may be included information necessary for calculating the structure factor based on the total scattering data such as a shape of a sample, arrangement, kinds of constituent elements, a composition, an absorption coefficient or the like.

The measurement data storage section 330 stores the measurement data acquired from the X-ray diffractometer 200. The measurement data includes the total scattering data. In addition to the total scattering data, may be included the information required for calculating the structure factor based on the total scattering data such as a type of a radiation source, a wavelength, a background, a shape of a sample, arrangement, kinds of constituent elements, a composition, an absorption coefficient or the like. In addition, when the background is low, the information required for calculating the structure factor may not include the background. The display section 340 displays the measurement data on the display device 520. Thus, the measurement data can be confirmed by a user. In addition, the user can perform instruction and designation for the control apparatus 300, the processing apparatus 400, and the like based on the measurement data.

### [Processing Apparatus]

The processing apparatus 400 is configured from a computer formed by connecting CPU, ROM, RAM and a memory to a bus. The processing apparatus 400 may be connected to the X-ray diffractometer 200 via the control apparatus 300.

The processing apparatus 400 comprises a structure factor acquiring section 410, a data converting section 420, and a scattering intensity calculating section 430. Each section can transmit and receive information via the control bus L. When the processing apparatus 400 is a separate configuration from the control apparatus 300, the input device 510 and the displaying device 520 are also connected to CPU of the processing apparatus 400 via an appropriate interface. In this case, the input device 510 and the display device 520 each may differ from one connected to the control apparatus 300.

The structure factor acquiring section 410 acquires the first structure factor based on the measured total scattering data. The structure factor acquiring section 410 may acquire the first structure factor calculated by another device based on the total scattering data actually measured by the X-ray diffractometer 200. The structure factor acquiring section 410 may acquire the first structure factor calculated by the structure factor calculating section 405, which is described later, based on the measured total scattering data.

The data converting section 420 separates the first structure factor into a short-range correlation and a long-range correlation. In one aspect, the data converting section 420 calculates PDF (Pair Distribution Function) from the first structure factor acquired by the structure factor acquiring section 410 and separates the first structure factor into a short-range correlation and a long-range correlation using PDF. The data converting section 420 may separate the first structure factor into short-range correlation and long-range correlation in a manner that does not use PDF.

In one aspect, the value of the boundary between the short-range correlation and the long-range correlation when the data converting section 420 separates the first structure factor into the short-range correlation and the long-range correlation is determined based on the size and shape of the region of the structural model.

The scattering intensity calculating section 430 acquires a structural model indicating the atomic arrangement in the finite region and calculates the short-range scattering intensity of the structural model. The scattering intensity calculating section 430 calculates a second structure factor from the short-range scattering intensity and the long-range correlation. The scattering intensity calculating section 430 may acquire a structural model generated by another device. The scattering intensity calculating section 430 may acquire a structural model generated by the structure estimating section 450 described later.

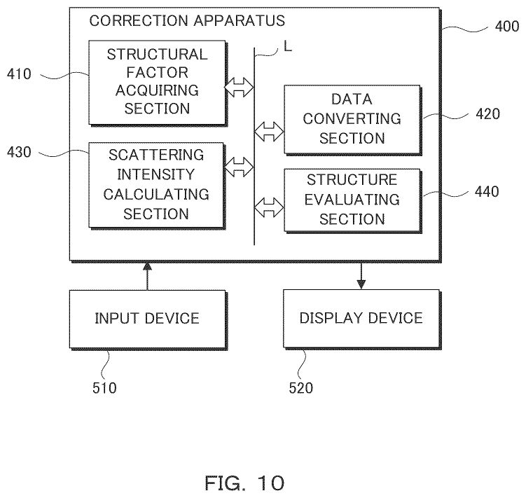

FIG. 10 is a block diagram showing a modified example of the configuration of the processing apparatus 400. As shown in FIG. 10, in one aspect, the processing apparatus 400 comprises a structure evaluating section 440. The structure evaluating section 440 calculates the degree of coincidence or deviation between the first structure factor and the second structure factor. Thus, how accurate the structural model reproduces the measured total scattering data can be confirmed.

The structure evaluating section 440 may calculate the degree of coincidence or deviation between the first structure factor and the second structure factor within a range equal to or larger than the lower limit value determined based on the value of the boundary between the short-range correlation and the long-range correlation.

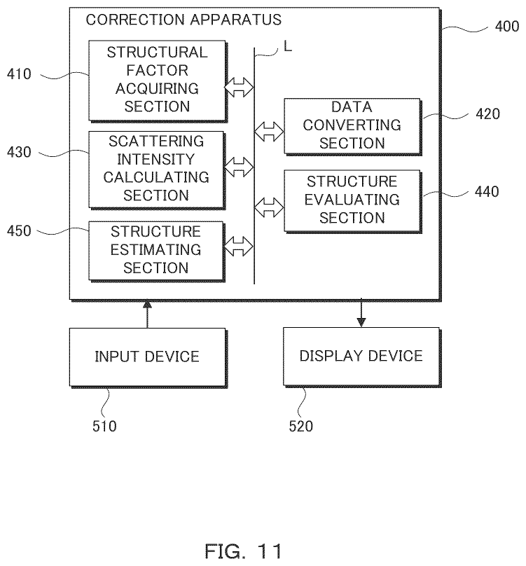

FIG. 11 is a block diagram showing a modified example of the configuration of the processing apparatus 400. As shown in FIG. 11, in one aspect, the processing apparatus 400 comprises the structure estimating section 450. The structure estimating section 450 generates a structural model. The structure estimating section 450 keeps a calculation region and may generate a structural model based on the size, shape, atomic arrangement and the like of the structural model. The size, shape, initial atomic arrangement and the like of the structural model may be designated by the user. In a case where the processing apparatus 400 comprises the structure estimating section 450, the structure evaluating section 440 may output a structural model in which the degree of coincidence or deviation satisfies a predetermined condition.

The structure estimating section 450 may generate a structural model by an RMC method.

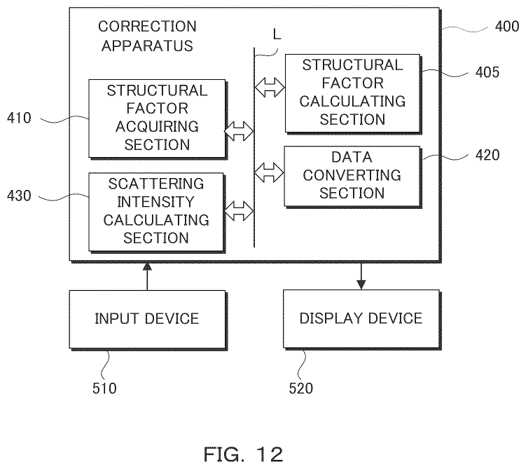

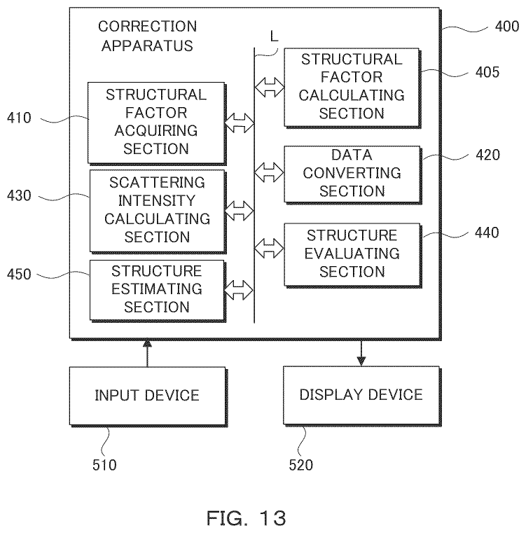

FIGS. 12 and 13 are block diagrams showing a modified example of the configuration of the processing apparatus 400. As shown in FIG. 12 or 13, in one aspect, the processing apparatus 400 comprises a structure factor calculating section 405. The structure factor calculating section 405 acquires the total scattering data of the sample and calculates the first structure factor based on the type of a radiation source, wavelength, background, shape of the sample, arrangement, kinds of constituent elements, a composition, and an absorption coefficient in the total scattering data. In addition, when the background is low, the structure factor may be calculated without using the background. When the processing apparatus 400 comprises the structure factor calculating section 405, the structure factor acquiring section 410 acquires the first structure factor calculated by the structure factor calculating section 405. In the block diagram of FIG. 10, a structure factor calculating section 405 may be further provided.

### [Measurement Method]

A sample S is installed in the X-ray diffractometer 200, and the goniometer is driven under a predetermined condition based on the control of the control apparatus 300. Further, X-rays are incident on the sample, and diffracted X-rays generated from the sample are detected. Thus, the diffraction data is acquired. The X-ray diffractometer 200 transmits the apparatus information, etc. and the acquired diffraction data as the measurement data to the control apparatus 300.

### [Processing Method]

### (Description of Flow Until Calculating the Second Structure Factor)

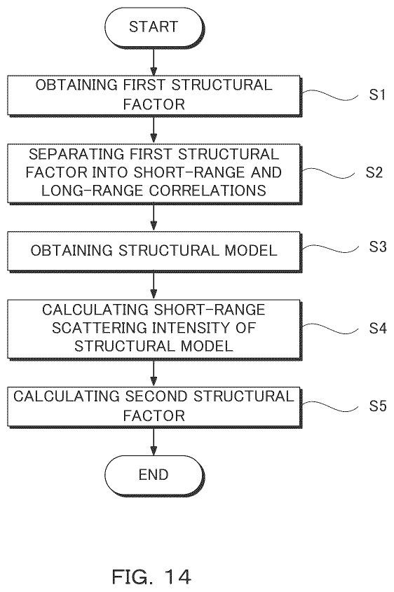

FIG. 14 is a flowchart showing an example of the operation of the processing apparatus 400. FIG. 14 shows an example of the operation until the second structure factor is calculated. First, the processing apparatus 400 obtains a first structure factor (step S1). Next, the first structure factor is separated into a short-range correlation and a long-range correlation (step S2). Next, a structural model is obtained (step S3). Next, the short-range scattering intensity of the structural model is calculated (step S4). Then, the second structure factor is calculated from the short-range scattering intensity and the long-range correlation (step S5), and the process ends. If necessary, a second structure factor or a structural model may be output. In this way, a second structure factor can be calculated that includes the total scattering data and the data of the structural model, and the second structure factor can be used to analyze both the total scattering data and the features of the structural model together.

### (Description of Flow Until Calculation of Degree of Coincidence or Deviation Between the First Structure Factor and the Second Structure Factor)

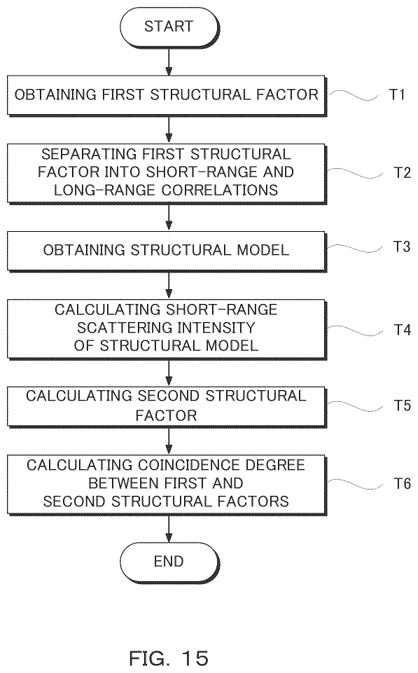

FIG. 15 is a flowchart showing a modified example of the operation of the processing apparatus 400. FIG. shows an example of the operation until the degree of coincidence or deviation between the first structure factor and the second structure factor is calculated. In the following description of the flowchart, the characteristic operation is described in detail, and the description of the operation already described may be omitted. From the acquiring of the first structure factor (step T1) to the calculating of the second structure factor (step T5) is similar to the step S1 to the step S5 described above. Then, the processing apparatus 400 calculates the degree of coincidence or deviation between the first structure factor and the second structure factor (step T6) and ends the calculation. If necessary, a second structure factor, a structural model, and a degree of coincidence or deviation between the first structure factor and the second structure factor may be output. Thus, how accurate the structural model reproduces the measured total scattering data can be confirmed.

(Description of Flow Until Outputting a Structural Model that Meets the Condition)

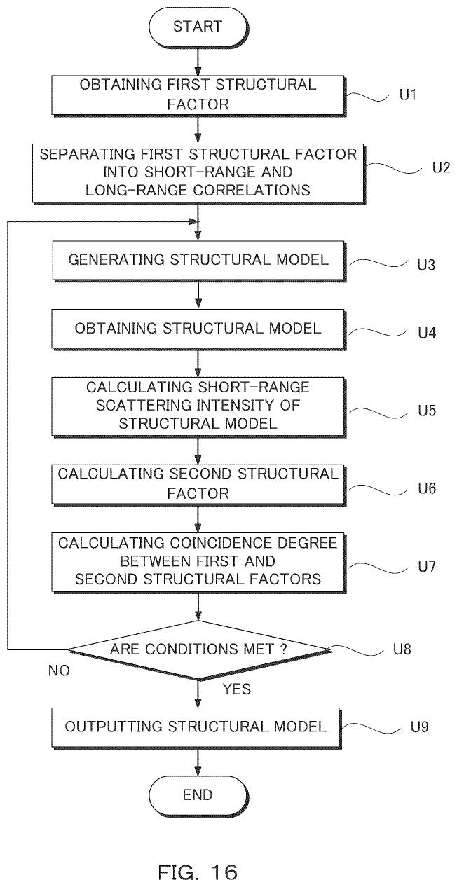

FIG. 16 is a flowchart showing a modified example of the operation of the processing apparatus 400. FIG. 16 shows an example of the operation until a structural model satisfying the condition is output. The acquiring of the first structure factor (step U1) and the separating of the first structure factor (step U2) are the same as the above-described steps. Next, a structural model is generated (step U3). The structural model may be generated by the processing apparatus 400 or may be generated by another apparatus or function.

From the acquisition of the structural model (step U4) to the calculation of the degree of coincidence or deviation between the first structure factor and the second structure factor (step U7) are the same as the above-described steps. Next, the processing apparatus 400 determines whether or not the degree of coincidence or deviation satisfies the set predetermined condition, and when the predetermined condition is not satisfied (step U8—NO), the operation returns to step U3 and the process up to step U7 are performed again. On the other hand, if the degree of coincidence or deviation satisfies the set predetermined condition (step U8—YES), the structural model is outputted (step U9) and the process ends. If necessary, the second structure factor or the degree of coincidence or deviation between the first structure factor and the second structure factor may be output. Thus, a structural model satisfying a predetermined condition can be generated and output.

Although the first structure factor is used as initial data in the above-described flowchart, the total scattering data may be used as initial data. In that case, the steps of acquiring total scattering data and calculating the first structure factor from the total scattering data may be included prior to the step of acquiring the first structure factor.

### Example

The system 100 configured as described above was used to measure the total scattering data of Ni. The structure factor and PDF were calculated using the total scattering data. Next, using the present methods, the generation of the structural model was repeated by RMC method until the degree of deviation between the first structure factor and the second structure factor became sufficiently small. Next, PDF was generated from the second structure factor when the degree of deviation satisfied the predetermined condition. Then, the degree of deviation between PDF generated from the first structure factor and PDF generated from the second structure factor was confirmed. The degree of deviation was confirmed using R_{P, G(r)} shown in the following Formula (16). wᵢ in Formula (16) is a weighting factor used as, for example, wᵢ=1/N. G_{obs} (r) represents a PDF generated from the first structure factor, and G_{cal} (r) represents a PDF generated from the structure factor including the data of the structural model. The degree of deviation R_{P, G(r)} is an index of which value decreases as the degree of similarity of the two PDF increases.

$${R}_{P , G ( r )} = \sqrt{\frac{{\sum }_{i}^{N} {{w}_{i} ( {G}_{obs} ( {r}_{i} ) - {G}_{cal} ( {r}_{i} ) )}^{2}}{{\sum }_{i}^{N} {{w}_{i} ( {G}_{obs} ( {r}_{i} ) )}^{2}}} \times 100 \qquad ( 16 )$$

Further, as a comparative example, the structural model was generated from the first structure factor by PDFgui which was the conventional method, and it was used to generate the structure factor and PDF. Then, the degree of deviation R_{P, G(r)} between PDF generated from the first structure factor and PDF generated with PDFgui was confirmed.

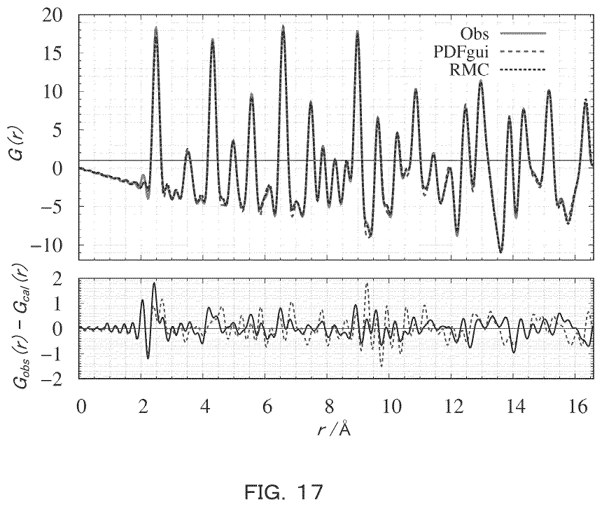

FIG. 17 is a graph showing PDF generated from the measured first structure factor, PDF generated from the second structure factor generated with the present method, and PDF generated with the comparative method and their respective residuals. It should be noted that Obs indicates a PDF generated from the first structure factor, that RMC indicates a PDF generated with the method of the present disclosure, and that PDFgui indicates a PDF generated with the method of the comparative example.

The degree of deviation R_{P, G(r)} of PDF generated with the method of the present disclosure was 6.55%. On the other hand, the degree of deviation R_{P, G(r)} of PDF generated with the method of the comparative example was 8.20%. Thus, it was confirmed that the method of the present disclosure can generate a PDF similar to the measured PDF than the method of the comparative example. In addition, it was confirmed that the structural model generated with the method of the present disclosure is a structural model that can explain the measured data more accurately than the structural model generated with the method of the comparative example.

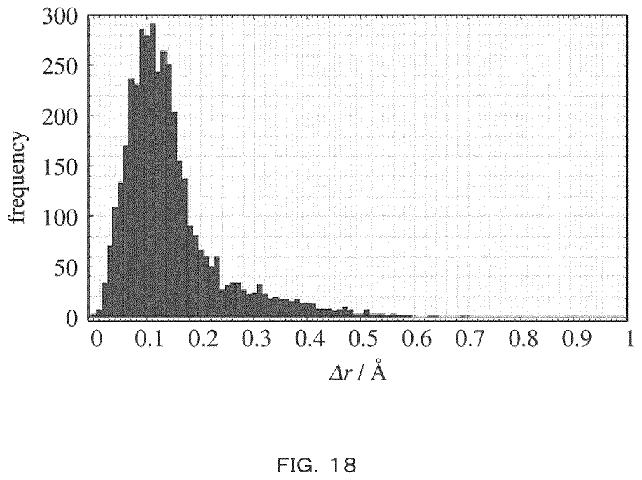

In addition, histograms and standard deviations of displacements calculated from the atomic arrangement of Ni before and after refinement by the method of the present disclosure and Rietveld analysis were measured. FIG. 18 is a histogram of displacement amounts calculated from a structural model generated with the method of the present disclosure. The standard deviation of the displacement was 0.0920 Å as calculated with the method of the present disclosure, whereas it was 0.0756 Å as calculated with Rietveld analysis. Thus, it was confirmed that the displacement calculated from the structural model generated with the method of the present disclosure was equivalent to the ones according to Rietveld analysis.

As a result, the processing apparatus, the system, the method, and the program of the present disclosure can make the parameters easily set by the user and reduce the calculation cost when the local structure of the sample is estimated. Also, both the features of the total scattering data and the structural model can be analyzed together. Furthermore, it is possible to generate an accurate structural model that can explain the measured data.

The present disclosure is not limited to the above-described embodiments. The scope of the present disclosure covers various modifications and equivalents included in the technical idea of the present disclosure. In addition, the names, structures, shapes, numbers, positions, sizes, and the like of the constituent elements shown in the drawings are for convenience of explanation and may be changed as appropriate.

What is claimed is:

1. A processing apparatus for processing a structure factor comprising:
processing circuitry configured to
acquire a first structure factor based on measured total scattering data,
separate the first structure factor into a short-range correlation and a long-range correlation, and
acquire a structural model indicating an atomic arrangement in a finite region, calculate a short-range scattering intensity of the structural model and calculate a second structure factor from the short-range scattering intensity and the long-range correlation.

2. The processing apparatus according to claim 1,
wherein a value of a boundary between the short-range correlation and the long-range correlation is determined based on a size and a shape of a region of the structural model.

3. The processing apparatus according to claim 1, wherein the processing circuitry is further configured to
calculate a degree of coincidence or a degree of deviation between the first structure factor and the second structure factor.

4. The processing apparatus according to claim 3, wherein the processing circuitry is further configured to
create the structural model, and
output the structural model in which the degree of coincidence or the degree of deviation satisfies a predetermined condition.

5. The processing apparatus according to claim 3,
wherein the processing circuitry is further configured to
calculate the degree of coincidence or deviation between the first structure factor and the second structure factor within a range equal to or greater than a lower limit value determined based on the value of the boundary between the short-range correlation and the long-range correlation.

6. The processing apparatus according to claim 4,
wherein the processing circuitry is further configured to
generate the structural model by a Reverse Monte Carlo (RMC) method.

7. The processing apparatus according to claim 1, wherein the processing circuitry is further configured to
acquire total scattering data of the sample and calculate the first structure factor based on a type of a radiation source, a wavelength, a background, a shape of the sample, an arrangement, kinds of a constituent element, a composition, and an absorption coefficient of the total scattering data, and
acquire the first structure factor.

8. A system comprising an X-ray diffractometer comprising an X-ray source, a detector for detecting X-rays, a goniometer for controlling the rotation of the sample, and the processing apparatus according to claim 1.

9. A method for processing a structure factor, the method comprising the steps of:
acquiring a first structure factor based on measured total scattering data,
separating the first structure factor into a short-range correlation and a long-range correlation,
acquiring a structural model representing an atomic arrangement in a finite region,
calculating a short-range scattering intensity of the structural model, and
calculating a second structure factor from the short-range scattering intensity and the long-range correlation.

10. A non-transitory computer-readable storage medium storing computer-readable instructions thereon which, when executed by a computer, cause the computer to perform a method, the method comprising:
acquiring a first structure factor based on measured total scattering data,
separating the first structure factor into a short-range correlation and a long-range correlation,
acquiring a structural model representing an atomic arrangement in a finite region,
calculating a short-range scattering intensity of the structural model, and
calculating a second structure factor from the short-range scattering intensity and the long-range correlation.

11. The method of claim 9, wherein a value of a boundary between the short-range correlation and the long-range correlation is determined based on a size and a shape of a region of the structural model.

12. The method of claim 9, further comprising:
calculating a degree of coincidence or a degree of deviation between the first structure factor and the second structure factor.

13. The method of claim 12, further comprising:
creating the structural model, and
outputting the structural model in which the degree of coincidence or the degree of deviation satisfies a predetermined condition.

14. The method of claim 12, further comprising:
calculating the degree of coincidence or deviation between the first structure factor and the second structure factor within a range equal to or greater than a lower limit value determined based on the value of the boundary between the short-range correlation and the long-range correlation.

15. The method of claim 13, further comprising:
generating the structural model by a Reverse Monte Carlo (RMC) method.

16. The method of claim 9, further comprising:
acquiring total scattering data of the sample and calculate the first structure factor based on a type of a radiation source, a wavelength, a background, a shape of the sample, an arrangement, kinds of a constituent element, a composition, and an absorption coefficient of the total scattering data, and
acquiring the first structure factor.
